(19) United States
(12) Patent Application Publication
(10) Pub. No.: US 2021/0085966 A1
(43) Pub. Date: Mar. 25, 2021

(54) IMPLANTABLE MEDICAL LEAD INCLUDING FIXATION RIDGE

(71) Applicant: MEDTRONIC, INC., Minneapolis, MN (US)

(72) Inventors: Walter Baxter, Minneapolis, MN (US); Jeff Bodner, Minneapolis, MN (US)

(73) Assignee: MEDTRONIC, INC., Minneapolis, MN (US)

(21) Appl. No.: 17/029,510

(22) Filed: Sep. 23, 2020

Related U.S. Application Data
(60) Provisional application No. 62/904,965, filed on Sep. 24, 2019.

(51) Int. Cl. A61N 1/05

(52) U.S. Cl. CPC A61N 1/0558

(57) ABSTRACT

An implantable medical lead with a fixation ridge is disclosed. The implantable medical lead includes a lead body extending between a proximal end and a distal portion having a distal end. The lead body includes lead body outside diameter. A conductor is disposed within the lead body and extends along a length of the lead body. An electrode is coupled to the conductor, and the electrode disposed on the lead body at the distal portion. A fixation mechanism comprising a ridge is disposed on the lead body at the distal portion, the fixation mechanism includes a fixation mechanism outside diameter that is greater than the lead body outside diameter.

### CROSS-REFERENCE TO RELATED APPLICATION

This Non-Provisional Utility application claims benefit to U.S. Provisional Application No. 62/904,965, filed Sep. 24, 2019, titled “IMPLANTABLE MEDICAL LEAD INCLUDING FIXATION RIDGE” the entirety of which incorporated herein by reference.

### BACKGROUND

The present disclosure relates generally to a method and apparatus that allows for electrical stimulation of body tissue, particularly sacral nerves. More specifically, this present disclosure relates to an implantable medical electrical lead having a stimulation electrode adapted to be implanted near the sacral nerves for stimulation of a bundle of sacral nerve fibers and a fixation mechanism for providing chronic stability of the stimulation electrode and lead. Moreover, the present disclosure relates to the method of implantation and anchoring of the medical electrical lead electrodes in operative relation to a selected sacral nerve to allow for stimulation.

Pelvic floor disorders such as urinary incontinence, urinary urge/frequency, urinary retention, pelvic pain, bowel dysfunction (constipation, diarrhea), and erectile dysfunction are bodily functions influenced by the sacral nerves. Specifically, urinary incontinence is the involuntary control over the bladder that is exhibited in various patients. Incontinence is primarily treated through pharmaceuticals and surgery. Pharmaceuticals may not adequately resolve the issue and can cause unwanted side effects, and a number of the surgical procedures have a low success rate and are not reversible. Several other methods have been used to control bladder incontinence, for example, vesicostomy or an artificial sphincter implanted around the urethea. These solutions also have drawbacks. In addition, some disease states do not have adequate medical treatments.

The organs involved in bladder, bowel, and sexual function receive much of their control via the second, third, and fourth sacral nerves, commonly referred to as S2, S3 and S4 respectively. Electrical stimulation of these various nerves has been found to offer some control over these functions. Several techniques of electrical stimulation may be used, including stimulation of nerve bundles within the sacrum. The sacrum, generally, is a large, triangular bone situated at the lower part of the vertebral column, and at the upper and back part of the pelvic cavity. The spinal canal runs throughout the greater part of the sacrum. The sacrum is perforated by the anterior and posterior sacral foramina that the sacral nerves pass through.

Neurostimulation leads have been implanted on a temporary or permanent basis having a stimulation electrode (at least one stimulation electrode) positioned on and near the sacral nerves to provide partial control for bladder incontinence. Temporary sacral nerve stimulation is accomplished through implantation of a temporary neurostimulation lead extending through the skin and connected with a temporary external pulse generator as described for example in commonly assigned U.S. Pat. Nos. 5,957,965 and 6,104,960. A permanent neurostimulator is implanted if stimulation is efficacious and it is possible to do so in the particular patient. Permanent implantation is accomplished by implanting a permanent neurostimulation lead, extending the proximal portion of the lead body subcutaneously, and connecting its proximal end with an implantable pulse generator, or IPG, implanted subcutaneously.

For example, U.S. Pat. Nos. 4,771,779, 4,607,739 and 4,739,764 disclose implanting an electrode on at least one nerve controlling the bladder and applying nerve stimulation energy to the nerve through the electrode. Electrodes positioned within the sacrum to control bladder function are also disclosed in U.S. Pat. No. 4,569,351.

In one example, a lead bearing a distal stimulation electrode is percutaneously implanted through the dorsum and the sacral foramen (a singular foramina) of the sacral segment S3 for purposes of selectively stimulating the S3 sacral nerve. The lead is advanced through the lumen of a hollow spinal needle extended through the foramen, the single distal tip electrode is positioned adjoining the selected sacral nerve. Stimulation energy is applied through the lead to the electrode to test the nerve response. The electrode is moved back and forth to locate the most efficacious location, and the lead is then secured by suturing the lead body to subcutaneous tissue posterior to the sacrum and attached to the output of a neurostimulator IPG. Despite the suture fixation, sacral nerve stimulation leads having a single discrete tip electrode can be dislodged from the most efficacious location due to stresses placed on the lead by an ambulatory patient. Surgical intervention can then be applied to reposition the electrode and affix the lead.

The current lead designs used for permanent implantation to provide sacral nerve stimulation through a foramen have several, e.g., four, of ring-shaped, stimulation electrodes spaced along a distal segment of the lead body adapted to be passed into or through the foramen along a selected sacral nerve. Each distal stimulation electrode is electrically coupled to the distal end of a lead conductor within the elongated lead body that extends proximally through the lead body. The proximal ends of the separately insulated lead conductors are each coupled to a ring-shaped connector element in a proximal connector element array along a proximal segment of the lead body that is adapted to be coupled with the implantable neurostimulation pulse generator, or neurostimulator IPG.

The electrode array is moved back and forth with respect to the sacral nerve while the response to stimulation pulses applied through one or more of the electrodes is determined. The IPG is programmed to deliver stimulation pulse energy to the electrode providing the optimal nerve response, and the selection of the electrodes can be changed if efficacy using a selected electrode fades over time due to dislodgement or other causes.

Electrical stimulation pulses generated by the neurostimulator IPG are applied to the sacral nerve through the selected one or more of the stimulation electrodes in either a unipolar or bipolar stimulation mode. In one unipolar stimulation mode, the stimulation pulses are delivered between a selected active one of the stimulation electrodes and the electrically conductive, exposed surface of the neurostimulator IPG housing or can that provides a remote, indifferent, or return electrode. In this case, efficacy of stimulation between each stimulation electrode and the neurostimulator IPG can electrode is tested, and the most efficacious combination is selected for use. In a further unipolar stimulation mode, two or more of the stimulation electrodes are electrically coupled together providing stimulation between the coupled together stimulation electrodes and the return electrode.

In a bipolar stimulation mode, one of the distal stimulation electrodes is selected as the indifferent or return electrode. Localized electrical stimulation of the sacral nerve is effected between the active stimulation electrode or electrodes and the indifferent stimulation electrode.

A issue associated with implantation of permanent and temporary neurostimulation leads involves maintaining the discrete ring-shaped electrode or electrodes in casual contact, that is in location where slight contact of the electrode with the sacral nerve may occur or in close proximity to the sacral nerve to provide adequate stimulation of the sacral nerve, while allowing for some axial movement of the lead body.

Typically, physicians spend a great deal of time with the patient under a general anesthetic placing the leads due to making an incision exposing the foramen and due to the difficulty in optimally positioning the small size stimulation electrodes relative to the sacral nerve. The patient is exposed to dangers associated with extended periods of time under a general anesthetic. Movement of the lead, whether over time from suture release or during implantation during suture sleeve installation, is to be avoided. Also, unintended movement of any object positioned proximate a nerve may cause unintended nerve damage. Moreover, reliable stimulation of a nerve entails consistent nerve response to the electrical stimulation that, in turn, entails consistent presence of the stimulation electrode proximate the sacral nerve. But, too close or tight a contact of the electrode with the sacral nerve can also cause inflammation or injury to the nerve diminishing efficacy and possibly causing patient discomfort.

Once the optimal electrode position is attained, the lead body is fixed to retard lead migration and dislodgement of the electrodes from the optimal position employing sutures or a sacral lead fixation mechanisms of the types described in the above-referenced U.S. Pat. No. 4,569,351 or an improved fixation mechanism of the type described in commonly assigned U.S. Pat. No. 5,484,445. However, it is desirable to avoid use of complex fixation mechanisms that include surgical exposure large enough to implant the fixation mechanism, in these cases requiring exposure and attachment to the sacrum.

Once fixation is completed, the proximal lead body is typically bent at about 90° and tunneled subcutaneously to a remote site where its proximal connector elements are coupled to the neurostimulator IPG which is then implanted at the remote site. In this process some axial and lateral dislodgement of the stimulation electrodes can also occur.

It is generally desirable to minimize surgical trauma to the patient through surgical exposure of the tissue and sacrum and use of sutures or fixation mechanisms to hold the electrodes in place. It is preferred to employ a minimally invasive, percutaneous approach in a path extending from the skin to the foramen that the neurostimulation lead is extended through.

The above-referenced U.S. Pat. No. 5,957,965 describes one such percutaneous approach for implantation of a temporary neurostimulation lead that extends through the patient's skin and is attached to an external pulse generator. Typically, the external pulse generator and exposed portion of the lead body are taped to the skin to inhibit axial movement of the lead body. When a stimulation time period ends, the lead is removed through the skin by application of traction to the exposed lead body, and the incision is closed. The neurostimulation lead bodies of U.S. Pat. No. 5,957,965 are formed with surface treatment or roughening in a portion proximal to the neurostimulation electrode expected to extend from the foramen to the patient's skin that is intended to increase the resistance to unintended axial dislodgement of the lead body to stabilize the electrode. A length of the lead body is formed with indentations or spiral ridges or treated to have a macroscopic roughening.

The prior art discloses a number of configurations of implantable medical electrical leads other than neurostimulation leads that employ fixation mechanisms to maintain a stimulation electrode in relation to a body organ or tissue. Cardiac pacing leads are commonly provided with passive fixation mechanisms that non-invasively engage heart tissue in a heart chamber or cardiac blood vessel or active fixation mechanisms that invasively extend into the myocardium from the endocardium or epicardium. Endocardial pacing leads having pliant tines that provide passive fixation within interstices of trabeculae in the right ventricle and atrial appendage are well known as exemplified by U.S. Pat. Nos. 3,902,501, 3,939,843, 4,033,357, 4,236,529, 4,269,198, 4,301,815, 4,402,328, 4,409,994, and 4,883,070, for example. Such tined leads typically employ three or four tines that extend outwardly and proximally from a band proximal to a distal tip pace/sense electrode and that catch in natural trabecular interstices when the distal tip electrode is advanced into the atrial appendage or the ventricular apex. Certain spinal cord stimulation leads have been proposed employing tines and/or vanes as stand-offs to urge the stimulation electrode in the epidural space toward the spinal cord as disclosed in U.S. Pat. Nos. 4,590,949 and 4,658,535, for example, and to stabilize the stimulation electrode in the epidural space as disclosed in U.S. Pat. No. 4,414,986, for example,

In the case of the above-referenced U.S. Pat. No. 3,939,843 that was directed to atrial tined leads, longitudinally extending rows of elongated tines were provided within a 270° arc extending away from a distal tip electrode canted in the remaining 90° section. The multiple rows of tines were intended to lodge in the trabecular interstices and force the canted tip against the atrial endocardial wall. However, it was found in practice that the canted tip is unnecessary and that only three, much shorter, tines in the 270° arc or four tines spaced apart by 90° in a common circumference like a ventricular tined lead, are sufficient. The rows of tines shown in the '843 patent are necessarily closely spaced because of the small area of trabeculae in the right atrium, and more proximal tines simply typically do not engage anything and make it difficult to lodge any of the tines in the interstitial spaces.

Such endocardial tined leads are typically introduced from a puncture site of the venous system to the cardiac site employing a stiffening stylet with a tip that can be formed with a curve extended down the lead lumen to advance it through the venous system and into the heart chamber. Percutaneous lead introducers are used to access the puncture site. The tines fold against the introducer lumen and the vein wall after the lead distal end exits the introducer lumen. However, the above-referenced '070 patent does describe thin, webbed, or scalloped tines adapted to be introduced through an introducer already advanced all the way into a heart chamber.

In certain cases, sensing leads that include electrogram (EGM) sense electrodes are implanted subcutaneously and coupled to implantable cardiac monitors. One such cardiac monitoring system as shown in U.S. Pat. No. 5,313,953, for example, employs elongated electrode supports at the distal ends of leads that are tunneled subcutaneously into a sense electrode array and affixed with sutures sewn through pre-formed suture holes to subcutaneous tissue. First and second rows of projections also extend outwardly and proximally from the opposed first and second side walls of the electrode supports whereby the projections are in a common plane with the sense electrode. It is expected that tissue growth will occur around the projections and stabilize the lead. However, it can also be seen that the leads are advanced subcutaneously between tissue layers expanded by tunneling. The projections would likely not engage tissue during the acute phase before they are encapsulated. For this reason, the elongated leads are sutured in place to maintain the electrode position until the projections are encapsulated by tissue ingrowth. Furthermore, it is less critical to maintaining exact positioning of the sense electrodes of such a subcutaneous cardiac monitor than maintaining stimulation electrodes in the implanted positions for cardiac or nerve or muscle stimulation because minute stimulation electrode movement can decrease efficacy and increase battery energy consumption or fail.

U.S. Pat. No. 6,999,819 describes a permanently implantable lead comprising a distal electrode that is maintained at a site in the body by a fixation mechanism that is formed on or integrally with the lead proximal to the electrode that is adapted to be implanted in and engage subcutaneous tissue, particularly muscle tissue, to inhibit axial movement of the lead body and dislodgement of the stimulation electrode. The fixation mechanism comprises a plurality M of tine elements arrayed in a tine element array along a segment of the lead proximal to the stimulation electrode array. Each tine element comprises at least N flexible, pliant, tines, each tine having a tine width and thickness and extending through a tine length from an attached tine end to a free tine end. The attached tine end is attached to the lead body from a tine attachment site and supports the tine extending radially from the lead body and proximally toward the lead proximal end. The M×N tines are adapted to be folded inward against the lead body when fitted into and constrained by the lumen of an introducer such that the tine length is folded up against lead body, and the folded tines do not overlap one another.

Accordingly, there remains a need in the art for a permanently implantable electrical sacral nerve stimulation lead that is capable of being passed percutaneously over a guide wire, and/or through the lumen of an introducer from the patient's skin to locate stimulation electrodes in casual contact with a sacral nerve, that provides acute fixation with muscle and tissue layers posterior to the sacrum, and that can be bent to extend subcutaneously to the neurostimulator IPG without disturbing the fixation so that the stimulation electrodes are less likely to be dislodged during the acute recovery phase and the chronic implantation period.

### SUMMARY

In one example, the present disclosure includes an implantable medical lead. The implantable medical lead includes a lead body extending between a proximal end and a distal portion having a distal end. The lead body includes lead body outside diameter, such as a lead body outside diameter at the distal portion. A conductor is disposed within the lead body and extends along a length of the lead body. An electrode is coupled to the conductor, and the electrode disposed on the lead body at the distal portion. A fixation mechanism comprising a ridge is disposed on the lead body at the distal portion, the fixation mechanism includes a fixation mechanism outside diameter that is greater than the lead body outside diameter. In one example, the implantable medical lead is included in an implantable medical system such as an implantable medical system having an implantable medical device.

Traditional fixation structures do not always provide fixation at the electrodes where fixation is desired. When fixation structures are inadvertently implanted in mobile fat or skin, there is a possibility of lead tip migration when the patient undergoes activities of daily living. Finally, implanting large fixation tines in the throat or tongue are can be problematic due to the ability to irritate or possible cause erosion of surrounding tissue including skin. The fixation mechanism with ridges of the present disclosure can obviate these complications by moving the fixation features proximate to the electrodes and better suiting the anatomy of surrounding muscle, fat, and skin.

The features of the implantable medical systems of the present disclosure are described with reference to sacral neuromodulation and implantable medical leads for use with sacral neuromodulation for illustration only. The implantable medical systems and implantable medical leads of the present disclosure can be configured and applied to as well as useful for other purposes, and other purposes can include such purposes as treatments in which electrodes are disposed in the tongue and throat and other locations of a patient.

### BRIEF DESCRIPTION OF THE DRAWINGS

FIG. 1 is a perspective view illustrating an example implantable medical system.

FIG. 2 is an exploded perspective view illustrating an implantable medical device useful with the system of FIG. 1.

FIG. 3 is a plan view illustrating an example implantable medical lead that can be used with the implantable medical system of FIG. 1.

FIG. 4 is a plan view illustrating a plurality of example implantable medical leads that can be used with implantable medical system of FIG. 1.

### DETAILED DESCRIPTION

Aspects of the present disclosure provide for implantable medical devices, methods of manufacturing such implantable medical devices, and implantable medical device systems including such implantable medical devices.

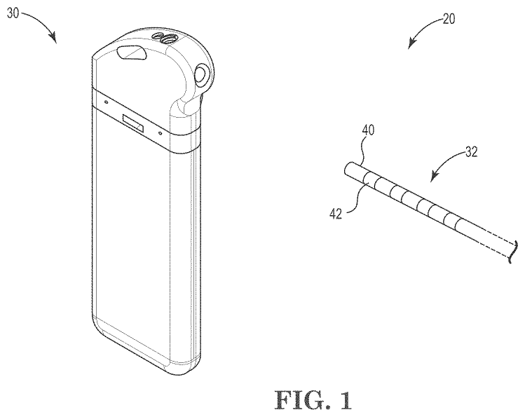

FIG. 1 illustrates an implantable medical device system 20. System 20 includes an implantable medical device (IMD) 30 and an implantable medical lead 32. In general terms, the implantable medical device 30 may be of various types, such as a device for producing electrical stimulation or for sensing physiological signals for various medical applications such as neurological or cardiac therapy. The implantable medical lead 32 includes a proximal end 40 of a lead body in which a series of electrical contacts 42 are located. Each electrical contact 42 has a corresponding conductor within the lead body that extends to a distal end (not shown) where a series of electrodes are present. During use, the proximal end 40 is inserted into the implantable medical device, establishing electrical interface between the electrical contacts 42 of the implantable medical lead 32 and electrical connectors carried by the implantable medical device 30. Stimulation signals generated by the implantable medical device 30 are delivered to the distal end of the implantable medical lead 32 and to targeted tissue and/or signals sensed by the distal end of the implantable medical lead 32 at the targeted tissue are delivered to the implantable medical device 30. The systems of the present disclosure can optionally include one or more additional components, such as one or more handset programmers configured and programmed to wirelessly interface with the implantable medical device 30.

In some examples, the system 20 and the implantable medical device 30 is configured to be useful or appropriate for providing stimulation therapy to a patient, and in particular sacral neuromodulation. In some examples, the system 20 can be described as an implantable programmable neuromodulation system that delivers electrical stimulation to the sacral nerve. Sacral neuromodulation therapy provided by the system 20 can be indicated for the management of the chronic intractable functional disorders of the pelvis and lower urinary or intestinal tract including overactive bladder, fecal incontinence, and nonobstructive urinary retention.

Sacral neuromodulation creates an electrical field near the sacral nerve to modulate the neural activity that influences the behavior of the pelvic floor, lower urinary tract, urinary and anal sphincters, and colon. The system 20 is configured to use current controlled stimulation to generate an electric field to modulate the sacral nerve. Electrical stimulation is delivered using metal electrodes provided with the implantable medical lead 32, which carry current in the form of electrons, to biological tissue, which carries current in the form of ions. An interface between the electrode and the tissue includes non-linear impedance that can be a function of the voltage across that interface. During current-controlled stimulation, an amount of current is regulated. The voltage is changed according to the actual value of impedance, such that changes in impedance will not affect the total amount of current delivered to the tissue. Current controlled waveforms can ensure that the electric field in the tissue is independent of electrode polarization or the voltage drop across the electrode-electrolyte interface. Alternatively, the systems of the present disclosure can be configured or programmed to use voltage-controlled stimulation.

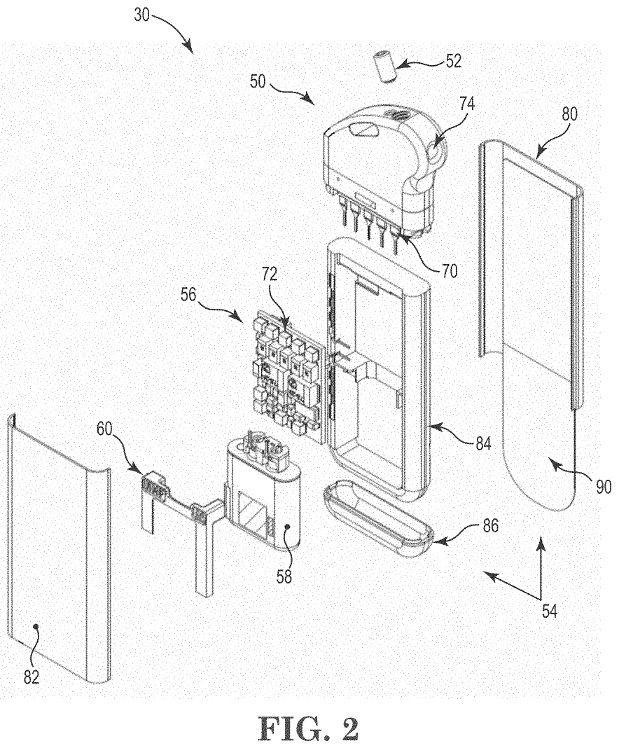

FIG. 2 illustrates an example of the implantable medical device 30 appropriate, for example, for generating the sacral neuromodulation therapy stimulation signals. The implantable medical device 30 can be configured to provide a small form factor, e.g., a volume on the order of approximately 3 cubic centimeters in some non-limiting embodiments, while generating desired stimulation signals over an extended lifetime. The implantable medical device 30 can serve as the power source of the sacral neuromodulation therapy described above in some embodiments. The smaller form factor or size as compared to conventional stimulation-type implantable medical devices can allow for a smaller implant location incision and smaller subcutaneous pocket, which may result in a more discreet implant. In some embodiments, the implantable medical device 30 incorporates various features described below that facilitate the small form factor size while providing desired performance attributes, such as remotely programmable stimulation signals or electrical pulses at therapy levels of interest, magnetic resonance imaging (MRI) compatibility, and remote charging.

In some embodiments, the implantable medical device 30 includes or defines a connector enclosure assembly 50, a set screw 52, a main enclosure assembly or can 54, electrical circuitry 56, a battery 58, and an optional desiccant assembly 60. Details on the various components are provided below. In general terms, the electrical circuitry 56, the battery 58 and the desiccant assembly 60 are maintained within the can 54. The battery 58 is electrically coupled to the electrical circuitry 56. The connector enclosure assembly 50 is assembled to the can 54, and includes one or more conductor fingers 70 that are electrically connected to individual circuitry components, and in particular contact pads 72, of the electrical circuitry 56. With this construction, electrical signals generated by the electrical circuitry 56 are delivered to the connector enclosure assembly 50 via the conductor fingers 70. The connector enclosure assembly 50 further forms or defines an entryway 74 sized to receive the proximal end 40 of the implantable medical lead 32. Electrical connectors provided with the connector enclosure assembly 50 interface with the electrical contacts 42 and are electrically connected to respective ones of the conductor fingers 70, thereby connecting the electrical circuitry 56 with implantable medical lead 32. The set screw 52 provides an electrical ground between the implantable medical lead 32 as inserted into the entryway 72 and the can 54.

The can 54 can assume various forms appropriate for maintaining the electrical circuitry 56 and the battery 58, as well as for assembly with the connector enclosure assembly 50. In some embodiments, the can 54 includes opposing shield bodies 80, 82, an insulator cup 84 and an end cap 86. The shield bodies 80, 82 can be formed of a surgically safe, robust material, e.g., titanium, such as a titanium alloy 6A1-4V ELI alloy per ASTM F136, and collectively generate a sleeve, e.g., the shield bodies 80, 82 can be secured to one another by, for example, laser seam welding applied to the interfacing edges. The sleeve, in turn, defines an open volume sized and shaped to receive the insulator cup 84. To facilitate final construction, a pressure sensitive adhesive liner 90 can be provided with the first shield body 80 that is removed prior to assembly to the insulator cup 84. A bottom opening to the sleeve collectively defined by the shield bodies 80, 82 is closed by the end cap 86. The end cap 86 and the connector enclosure assembly 50 can be assembled (e.g., welded) to the shield bodies 80, 82 to provide a hermetically sealed case.

The insulator cup 84 serves as a chassis, sized and shaped to fit snugly between the shield bodies 80, 82. The insulator cup 84 spatially secures the electrical circuitry 56 and the battery 58 via appropriately sized and shaped cavities. The insulator cup 84 can be formed of an electrically non-conductive or insulative material, such as a polymer.

The electrical circuitry 56 can include various electrical components and connections appropriate for providing, in some non-limiting embodiments, a pulse generator for therapy stimulation, e.g., a constant current stimulation engine, sensing circuitry for measuring physiological parameters, telemetry for communication with external devices (e.g., inductive telemetry at 175 KHz), memory, and a recharge circuit in some non-limiting embodiments. For example, the electrical circuitry 56 can deliver stimulation signals to the contact pads 72, and can process or act upon sensed signals received at the contact pads 72. The electrical circuitry 56 optionally provides various stimulation signal parameters, for example current controlled amplitude with a resolution of 0.1 mA steps, an upper limit of 12.5 mA, and a lower limit of 0.0 mA; a rate of 3-130 kHz; pulse width increments of 10 μs steps with a maximum of 450 μs and a minimum of 20 μs.

The battery 58 can assume various forms appropriate for generating desired stimulation signals, and in some embodiments is a rechargeable battery. For example, the battery 58 can incorporate lithium ion (Li+) chemistry, although other battery constructions known in the art are also acceptable.

The desiccant assembly 60 is sized and shaped for mounting within the can 54, and provides or carries an appropriate desiccant material to promote a dry environment within the can 54.

The connector enclosure assembly 50 can be mounted to the can 54 in a hermetically sealed fashion. The conductor fingers 70 and the ground conductor 124 are arranged to extend to a corresponding one of the contact pads 72, and are welded, e.g., pressure gas welding. The desiccant assembly 60 can be placed into the can 54 following the welding process, or otherwise delayed until a remaining step is to add the second shield body 82. In this manner, the desiccant is exposed to the ambient conditions for only a short time prior to the interior of the can 54 being isolated from the exterior. This can preserve the effectiveness of the desiccant.

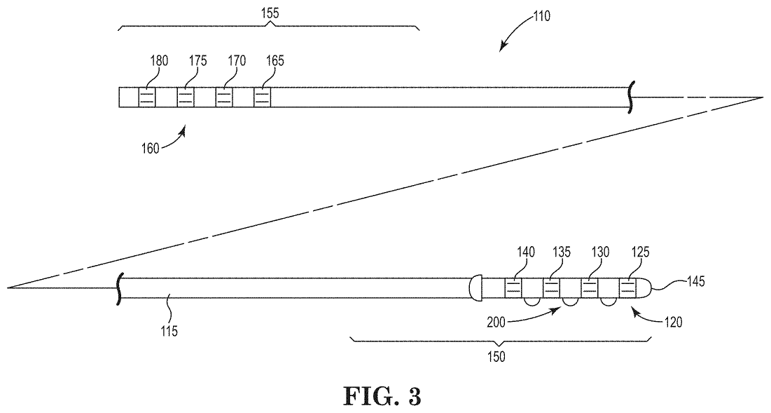

FIG. 3 illustrates an implantable medical lead 110, which can be an example of implantable medical lead 32, that may be applied to provide sacral nerve stimulation that allow for non-direct contact stimulation of the sacral nerves comprises a lead body 115 and a ring-shaped electrode, such as four ring-shaped electrode 125, 130, 135, and 140 in an electrode array 120 extending proximally from the lead distal end 145. As illustrated, the lead body 115 includes an outer surface. In one example, the outer surface is generally smooth along the length of the lead body 115. An electrode array can include one or more electrodes. For example, an implantable medical lead could include an electrode array with a single electrode disposed on the distal end of the lead body. Other configurations are contemplated. An outer diameter of the lead body 115, such as the outer diameter of the outer surface of the lead body 115, can be in the range of about 0.5 mm to about 2 mm, and the lead 110 can be of a suitable length, such as about 28.0 cm long. The electrode array 120 with four ring electrodes can extends proximally longitudinally for a length of about 25.0 mm from the distal end 145. In one example, the electrodes 125, 130, 135 and 140 are made of a solid surface, bio-compatible material such as a tube formed of platinum, platinum-iridium alloy, or stainless steel, of about 3.0 mm in length that does not degrade when electrical stimulation is delivered through it separated by shorter insulator bands.

Each stimulation electrode 125, 130, 135, and 140 is electrically coupled to the distal end of a coiled wire lead conductor within the elongated lead body 115 that extends proximally through a distal portion 150 and through a proximal portion 155 of the lead body 115. The proximal ends of the separately insulated lead conductors are each coupled to respective ring-shaped connector elements 165, 170, 175, and 180 in a proximal connector element array 160 along the proximal portion 155 of the lead body 115 adjacent the lead proximal end 185. The conductor wires can be formed of an MP35N alloy and are insulated from one another within an insulating polymer sheath such as polyurethane, fluoropolymer, or silicone rubber. An example diameter of the lead body 115 is 1.3 mm but smaller diameters are also contemplated. The lead conductor wires are separately insulated by an insulation coating and are wound in a quadra-filar manner having a common winding diameter within the outer sheath. The coil formed by the coiled wire conductors defines a lead body lumen of the lead body 115. In some examples, a further inner tubular sheath could be interposed within the aligned wire coils to provide the lead body lumen.

The connector elements 165, 170, 175, and 180 are adapted to be coupled with a neurostimulator IPG, such as implantable medical device 30, additional intermediate wiring, or other stimulation device adapted to be implanted subcutaneously. An example of such an implantable pulse generator is available under the trade designation Medtronic InterStim Neurostimulator from Medtronic, Inc. Electrical stimulation pulses generated by the implantable medical device 30 are applied to the sacral nerve through one or more of the stimulation electrodes 125, 130, 135 and 140 in a unipolar or bipolar stimulation mode.

The axial lead body lumen (not shown) extends the length of the lead body 115 between a lumen proximal end opening at lead proximal end 185 and a lumen distal end opening at lead distal end 145. A straight wire attached to the handle of a guide wire or stiffening stylet can be inserted through the lead body lumen to assist in implanting the lead 110.

A fixation mechanism 200 is formed on the lead body 115, such as on the outer surface of the lead body 115 as illustrated, in the distal lead portion 150 that is adapted to be implanted in and engage subcutaneous tissue to inhibit axial movement of the lead body 115 and dislodgement of the stimulation electrodes 125, 130, 135 and 140 in the example. For example, the fixation mechanism can be formed on the outer surface of the lead body 115 in the distal portion within 25 mm-30 mm of the distal tip. In one example, the fixation mechanism 200 is disposed within the electrode array 120. In another example, the fixation mechanism can be disposed within and proximal to the electrode array 120. In still another example, the fixation mechanism 200 can disposed distal to the electrode array such as on the lead distal end 145. In still another example, the fixation mechanism 200 can be disposed to be completely proximal to the electrode array 120. Other configurations are contemplated. Further, the fixation mechanism 200 can be applied in conjunction with another fixation mechanism such as an array of tines that include a larger outside diameter. The array of tines can be disposed proximal to the fixation mechanism 200

The fixation mechanism 200 comprises a ridge disposed on the lead body 115. For example, the fixation mechanism 200 includes a plurality of discrete ridges disposed on the lead body 115. The ridges can be formed of a material different from the outer surface of the lead body 115. The fixation mechanism 200 extends or enlarges the outer diameter of the implantable medical lead 110 from the outer diameter of the lead body 115 at the distal portion 150. The ridges may be in the form of dots, bumps, or bands disposed on the distal portion 150 of the lead body 115. For instance, the ridges can be formed of a single, discrete, spaced apart dot or bump on the outer surface of the lead body that does not touch another ridge on the outer surface of the lead body. In one example, the ridges are formed on a side of the outer surface of the lead body 115. In another example, the ridges encircle the outer diameter of the lead body 115. The ridges can extend the outer diameter of the implantable medical lead by approximately 1 mm or less. In one example, the ridges are of a size and shape to less than double the outside diameter of the implantable medical lead at the ridge than at the underlying lead body 115. In one example, the ridges can extend along the length of the lead body 115 by approximately 1 mm-2 mm or less. In one example, a ridge is included in the distal portion within 10 mm of the distal tip 145.

The fixation mechanism 200 provides the distal portion 150 and lead distal end 145 with stability not offered with tines or other fixation structures proximal to the electrode array 120. For example, fixation structures proximal to the electrode array 120 are often offset from the lead distal end 145 by approximately 25 mm and are intended to be disposed in muscle. Tines can displace tissue. Often, tissue proximate the muscle, such as skin or fat, can move and the tines can be placed in the skin or fat, and the tines can be less effective at providing stability in such tissue. Tissue proximate the lead distal end 145 of an implanted implantable medical lead 110 is denser relative to tissue more proximal to the lead distal end. A subtle fixation mechanism, such as fixation mechanism 200 with ridges, providing a slight increase to the outside diameter of the implantable medical lead 110 over the outside diameter of the lead body 115 at the distal portion 150, provides suitable stability to the distal portion 150 and the lead distal end 145.

In one example, the fixation mechanism 200 can be applied to a constructed lead body 115 or partially constructed lead body 115. For example, the fixation mechanism can be attached, molded, or adhered to the lead body 115. The fixation mechanism 200 can be constructed separately from the lead body 115 and adhered to the outside of the constructed lead body 115. The fixation mechanism 200 can include a plurality of insulative elements attached, such as adhered, to the lead body 115 at the distal portion 150. In another example, the fixation mechanism 200 can include a plurality of insulative sleeves disposed around the lead body 115 and attached to the lead body 115. In another example, the ridges may be “dripped on,” “painted on” or otherwise deposited onto the lead body 115 at selected locations on the distal portion 150. Materials used to form the ridges can include polyurethane, silicone, other insulative materials, or in some instances, other materials including conductive materials disposed away from and not in contact with the electrodes 125, 130, 135, 140.

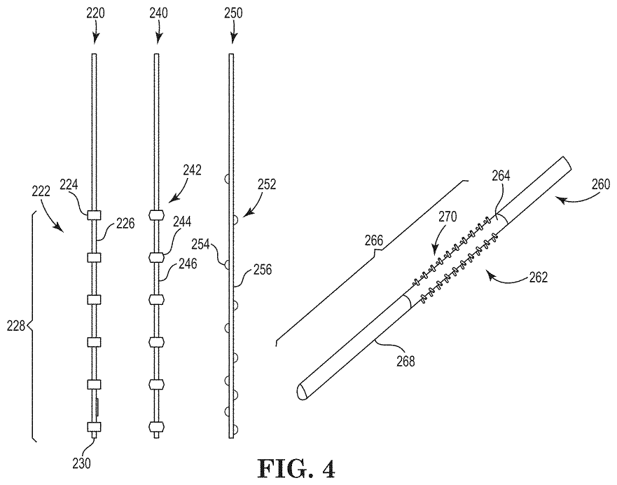

FIG. 4 illustrates a plurality of examples of implantable medical leads having a fixation mechanism such as fixation mechanism 200. For example, implantable medical lead 220 includes fixation mechanism 222 as a plurality of ridges 224 in the form of cylindrical sleeves disposed about lead body 226 and along an axial length of lead body 226 in a distal portion 228. The distal portion 228 can include the portion of the lead body 226 within 25 mm of the lead distal end 230 of the lead body 226. An electrode array can be disposed in between the ridges 224 along the length of the distal portion 228. The length of each ridge can be a fraction of the outside diameter of each sleeve, and the outside diameter of each sleeve can be less than double the outside diameter of the lead body 226. The ridges 224 can be of the same or a varied outside diameter with respect to each other and can be of the same or a varied axial length with respect to each other. The ridges 224 are illustrated as spaced equidistant from each other along the axial length of lead body. In an alternate example, the ridges are spaced at variable distances from each other, such as ridges disposed proximate the lead distal end 230 may be spaced closer to each other than ridges disposed away from the lead distal end.

Implantable medical lead 240 includes fixation mechanism 242 as a plurality of ridges 244 in the form of shaped sleeves disposed along an axial length of the lead body 246 and about the lead body 246. The shaped sleeves of ridges 244 can include a relatively smaller outside diameter near the edges of the sleeve and a relatively larger outside diameter between the edges of the sleeve. The sleeve can include a peak between the edges as illustrated or may include a smooth change in outside diameter along the length of the sleeve.

Implantable medical lead 250 includes fixation mechanism 252 as a plurality of ridges 254 in the form of dots disposed along an axial length of the lead body 256. The dots can be adhered to the lead body in the distal portion and near the lead distal end. The dots can be positioned at various radial locations around the lead body as illustrated.

Implantable medical lead 260 includes fixation mechanism 262 as a sleeve 264 attached to a distal portion 266 of a lead body 268. The sleeve 264 includes a miniature tine array 270 around the sleeve 264. The miniature tine array 270 can include a plurality of tines radially disposed around the sleeve 264 at a plurality of locations along the length of the sleeve 264. The sleeve 264 with the tine array 270 can be manufactured apart from the lead body 268 and later attached to form the implantable medical lead 260.

All patents and patent applications referenced in the disclosure are incorporated by reference in their entireties into this disclosure.

Although the present disclosure has been described with reference to preferred embodiments, workers skilled in the art will recognize that changes can be made in form and detail without departing from the spirit and scope of the present disclosure.

What is claimed is:

1. An implantable medical system comprising:
an implantable medical lead comprising:
a lead body extending between a proximal end and a distal end having a distal portion and a lead body outside diameter;
a conductor within the lead body extending along a length of the lead body;
an electrode coupled to the conductor, the electrode disposed on the lead body at the distal portion; and
a fixation mechanism comprising a ridge disposed on the lead body at the distal portion, the fixation mechanism having a fixation mechanism outside diameter that is greater than the lead body outside diameter.

2. The system of claim 1 wherein the distal portion includes a distal tip, wherein the electrode is disposed on the distal tip.

3. The system of claim 1 wherein the distal portion includes a distal tip, wherein the electrode is disposed proximal to the distal tip and the ridge is disposed distal to the electrode.

4. The system of claim 3 wherein the fixation member is disposed distal to the electrode.

5. The system of claim 1 wherein the fixation member includes a plurality of ridges disposed on the distal portion.

6. The system of claim 1 wherein the ridge includes a sleeve disposed around the lead body.

7. The system of claim 1 wherein the ridge includes a dot disposed on the lead body.

8. The system of claim 1 wherein the fixation mechanism outside diameter that is less than twice as large as the lead body outside diameter at the distal portion.

9. The system of claim 1 wherein the fixation mechanism includes a sleeve having a sleeve length, the sleeve having a miniature tine array disposed along the sleeve length.

10. The system of claim 1 comprising a plurality of electrodes coupled to the conductor at the distal portion.

11. The system of claim 1 comprising an implantable medical device operably coupled to the implantable medical lead.

12. An implantable medical lead comprising:
a lead body extending between a proximal end and a distal end having a distal portion and an lead body outside diameter;
a conductor within the lead body extending along a length of the lead body;
an electrode coupled to the conductor, the electrode disposed on the lead body at the distal portion; and
a fixation mechanism comprising a ridge disposed on the lead body at the distal portion, the fixation mechanism having a fixation mechanism outside diameter that is greater than the lead body outside diameter.

13. The implantable medical lead of claim 12 wherein the distal portion includes a distal tip, wherein the electrode is disposed on the distal tip.

14. The implantable medical lead of claim 12 wherein the distal portion includes a distal tip, wherein the electrode is disposed proximal to the distal tip and the ridge is disposed distal to the electrode.

15. The implantable medical lead of claim 14 wherein the fixation member is disposed distal to the electrode.

16. The implantable medical lead of claim 12 wherein the fixation member includes a plurality of ridges disposed on the distal portion.

17. The system of claim 12 wherein the ridge includes a sleeve disposed around the lead body.

18. The implantable medical lead of claim 12 wherein the ridge includes a dot disposed on the lead body.

19. The implantable medical lead of claim 12 wherein the fixation mechanism outside diameter that is less than twice as large as the lead body outside diameter at the distal portion.

20. The implantable medical lead of claim 12 wherein the fixation mechanism includes a sleeve having a sleeve length, the sleeve having a miniature tine array disposed along the sleeve length.

21. The implantable medical lead of claim 12 comprising a plurality of electrodes coupled to the conductor at the distal portion along an axial length.

22. The implantable medical lead of claim 21 wherein the ridge is disposed on the lead body between the electrodes along the axial length.
